(19) United States
(12) Patent Application Publication
(10) Pub. No.: US 2008/0279936 A1
(43) Pub. Date: Nov. 13, 2008

(54) Estradiol metabolites for reduction of endothelin production

(75) Inventors: Edwin K. Jackson, Pittsburgh, PA (US); Raghvendra K. Dubey, Glenshaw, PA (US)

Correspondence Address: THE MCCALLUM LAW FIRM, P. C., 685 BRIGGS STREET, PO BOX 929, ERIE, CO, 80516, US

(73) Assignee: The University of Pittsburgh

(21) Appl. No.: 10/268,091

(22) Filed: Oct. 10, 2002

Related U.S. Application Data
(60) Provisional application No. 60/327,770, filed on Oct. 10, 2001.

(51) Int. Cl. A61K 31/56; A61K 9/22

(52) U.S. Cl. 424/468; 514/182

(57) ABSTRACT

Methods are provided for the decrease of endothelin production in an individual. In particular, the methods include the reduction of endothelin production by the administration of a composition comprising an estradiol metabolite. Preferred estradiol metabolites include 2-methoxyestradiol, 4-methoxyestradiol, 2-hydroxyestradiol and 4-hydroxyestradiol or prod rugs thereof. The composition may be in the form of a controlled release formulation. The present methods are useful as a therapeutic for a wide variety of disease states.

### RELATED APPLICATIONS

This application claims priority from U.S. Provisional Application No. 60/327,770 filed Oct. 10, 2001.

The present invention was developed in part with Government support under grant numbers HL35009 and HL55314. The Government has certain rights in this invention.

### FIELD OF THE INVENTION

The present invention relates to methods of decreasing endothelin production and specifically to the administration of estradiol metabolites to decrease such endothelin production. More particularly, the present invention relates to the use of estradiol metabolites with little estrogenic activity such as 2-hydroxyestradiol, 4-hydroxyestradiol, 2-methoxyestradiol and 4-methoxyestradiol all of which may be delivered in a controlled release formulation for the treatment of disorders caused by increased endothelin production.

### BACKGROUND OF THE INVENTION

Endothelin, one of the most potent vasoconstrictors, was first discovered by Yanagisawa and co-workers in 1988. (See Yanagisawa, M., et al., Nature 332:411 (1988), the disclosure of which is incorporated herein by reference.) It was first isolated, characterized, and cloned in porcine aortic endothelial cells. (See id.). Endothelin has three isoforms, all closely related. The first, endothelin-1 (“ET-1”), is a 21-amino acid peptide that is present in many mammalian species, including humans. The other two endothelin isoforms, endothelin-2 and endothelin-3 are encoded by separate genes and have different structural characteristics. Although vascular endothelial cells are the major source of endothelins, including ET-1, it is also produced by a wide variety of cell types including renal tubular endothelium, glomerular mesangium, cardiac myocytes, glia, the pituitary, macrophages and mast cells. (See Inoue, A., et al., Proc. Natl. Acad. Sci. USA 86:2863 (1989), the disclosure of which is incorporated herein by reference.)

Endothelin-1 production has been associated with a wide variety of disease states. In various studies in dogs and rats it has been seen that endothelin peptides have both contractile and promitogenic actions in renal mesangial cells. (See Brooks, D. P., et al., J. Pharmacol. Experiments and Therapeutics 271:1 (1994), the disclosure of which is incorporated herein by reference.) In addition, ET-1 has also been shown to be capable of stimulating quiescent mesangial cells in order for them to re-enter G1-phase and proliferate. Endothelin-1 also increases mesangial DNA topoisomerase-1 activity by a pertussis toxin sensitive pathway. (See id.) In addition, endothelins also evoke arachidonic acid release and production of PGE-2 from mesangial cells. (See Rubanyi, G. M., et al., J. Pharmacol. Rev. 45:325 (1994), the disclosure of which is incorporated herein by reference.)

Endothelin-1 appears to play a role in the pathogenesis of acute renal failure after renal ischaemia as plasma levels of ET-1 are increased in patients with acute renal failure. (See Takahashi, K., et al., Nephron. 66:373 (1994), the disclosure of which is incorporated herein by reference.) Additionally, endothelin-1 causes potent vasoconstriction and prolonged elevation of blood pressure in experimental models. But the relationship between the plasma levels of endothelin-1 and severity of hypertension is inconsistent in humans. (See Saito, Y., et al., N. Enql. Jour. Med. 322:205 (1990), the disclosure of which is incorporated herein by reference.) Plasma endothelin levels were also found increased in a study of two patients with skin tumor-haemangioendothelioma, who had developed hypertension. (See Yokokawa, K., et al., Ann. Intern. Med. 114:213 (1991), the disclosure of which are incorporated herein by reference.)

Additionally, plasma endothelin levels are increased in animal models of chronic heart failure and in patients with chronic heart failure. In patients, increased plasma endothelin levels correlate closely with the degree of haemodynamic and functional impairment, with higher levels predicting a greater likelihood of death or need for cardiac transplantation. (See Wei, C. M., et al., Circulation 89:1580 (1994), and Wieczorek, I., et al., British Heart J. 72:436 (1994), the disclosures of which are incorporated herein by reference.) In the failing heart, production of endothelin-1 in vascular endothelial cells is enhanced by angiotensin-II, vasopressin, norepinephrine and by mechanical factors such as shear stress and endothelial stretching. (See id.).

Endothelin-1 also appears to play a role in ischaemic heart disease. In human studies, plasma endothelin levels are increased in unstable angina and acute myocardial infarction. (See Remiss, G., et al., Lancet 342:589 (1993), and Takahashi K., et al., Nephron. 66:373 (1994), the disclosures of which are incorporated herein by reference.) Patients with stable angina do not have increased endothelin levels. (See Watanaba, T., et al., Circulation Research 69:1573 (1991), the disclosure of which is incorporated herein by reference.) There is a direct correlation between a high plasma endothelin level and a poor prognosis of recovery. (See Omland, T., et al., Circulation 89:1573 (1994), the disclosure of which is incorporated herein by reference.)

In addition, patients with variant angina, such as Prinzmetal's angina, are known to have endothelial dysfunction affecting the L-arginine nitric oxide system, and as a potent vasoconstrictor of coronary arteries, endothelin-1 has been implicated in the pathophysiology of this condition. (See Willette, R. N., et al., J. Pharmacol. Experiments and Therapeutics 280:695 (1997), the disclosure of which is incorporated herein by reference.) Patients with variant angina also have increased prevalence of Raynaud's disease. In both these conditions, endothelins have been implicated as a causative factor. (See id.).

Endothelin-1 also appears to have a causative role in mediating subarachnoid hemorrhage (SAH)-induced vasospasm. Plasma and CSF endothelin levels are significantly increased in patients after SAH and plasma levels of endothelins are highest in those who develop vasospasm. (See Clozel, M., et al., Nature 365:759 (1993), the disclosure of which is incorporated herein by reference.)

In the recent studies it has also been found that levels of endothelins are increased during migraine headaches. Levels of endothelins have not been found to be higher between attacks or in patients with episodic or chronic tension headaches indicating that raised endothelin levels are due to migraine and not merely a response to the headache itself (See id.). The vasoconstriction associated with the first phase of migraine could be attributed at least in part to the release of vasoactive substances, such as the endothelins (See Haynes, W. G., et al., Circulation 93:1860 (1996), the disclosure of which is incorporated herein by reference.)

Endothelin-1 antagonists have been chemically synthesized and used in patients demonstrating many of the health conditions listed above in an attempt to produce a new therapeutic regime. Intravenous administration has proven effective in many of the conditions but, unfortunately, potent antagonists have not yet been found and a new therapeutic capable of decreasing ET-1 production is needed to help the many people suffering from conditions associated with an increase in ET-1 levels.

Citation of the documents herein is not intended as an admission that any is pertinent prior art. All statements as to the date or representation as to the contents of these documents is based on subjective characterization of information available to the applicant, and does not constitute any admission as to the accuracy of the dates or contents of these documents.

### SUMMARY OF THE INVENTION

Methods are provided for reducing endothelin production in an individual, comprising administering a therapeutically effective amount of a composition containing an estradiol metabolite in order to decrease endothelin production and alleviate the contribution of endothelin to a particular health condition. The present methods will provide a therapeutic option for the multitude of health problems associated with high endothelin levels where lower levels of endothelin will provide a treatment effect.

These and other objects of the invention are achieved by one or more of the following embodiments.

In one aspect, the invention features a method for reducing endothelin production in an individual, comprising: administering to the individual a therapeutically effective amount of a composition comprising an estradiol metabolite to cause a reduction in endothelin production in the individual.

In another aspect, the estradiol metabolite is selected from the group consisting of 2-hydroxyestradiol, 2-methoxyestradiol, 4-hydroxyestradiol and 4-methoxyestradiol.

In yet another aspect, the composition is a prodrug of the estradiol metabolite.

In a further aspect, the composition comprises a controlled release formulation.

In yet another aspect, the endothelin production is endothelin-1 production.

Other features and advantages of the invention will be apparent from the following description of the preferred embodiment, and from the claims.

### BRIEF DESCRIPTION OF THE DRAWINGS

The following drawings form part of the present specification and are included to further demonstrate certain embodiments. These embodiments may be better understood by reference to one or more of these drawings in combination with the detailed description of specific embodiments presented herein.

FIG. 1 shows time-dependent inhibitory effects of 2 nmol/L of estradiol (“1-Estradiol”), 2-hydroxyestradiol (“2-OHE”), and 2-methoxyestradiol (“2-MeOE”) on the release of endothelin-1 (“ET-1”) in the medium by porcine coronary artery endothelial cells (“PCAECs”) treated for various times in accordance with methods of the present invention. Values represent mean ±SEM from a representative experiment conducted in triplicate, and similar results were obtained in three independent experiments. *P<0.05 versus control cells treated with vehicle alone.

FIGS. 2A-2D show concentration-dependent inhibitory effects of estradiol (“1-Estradiol”), 2-hydroxyestradiol (“2-OHE”), and 2-methoxyestradiol (“2-MeOE”) on fetal calf serum ((“FCS”); 2.5%) (FIG. 2A); angiotensin II-stimulated ((“Ang II”); 100 nmol/L) (FIG. 2B); thrombin-stimulated ((“Thr”); 4 U/mL) (FIG. 2C); and TNF α-stimulated (10 ng/mL) (FIG. 2D) release of ET-1 in the medium of PCAECs treated for 4 hours. Values represent mean ±SEM from a representative experiment conducted in triplicate, and similar results were obtained in three independent experiments. *P<0.05 versus control cells treated with vehicle or the stimulatory agents alone; §P<0.05 versus inhibitory effects of estradiol.

FIG. 3 shows comparison of the inhibitory effects of 10 nmol/L of estradiol and various clinically used estrogens (17β-estradiol (“βE”); 17α-estradiol (“αE”); estradiol cypionate (“EC”); estradiol valerate (“EV”); estradiol benzoate (“EB”); estriol (“E3”); estrone (“E2”); and estrone sulfate (“ES”) in the presence and absence of ICI182780 (50 μmol/L) on 2.5% fetal calf serum-induced ET-1 release into the medium of cells stimulated for 4 hours in accordance with methods of the present invention. Values represent mean ±SEM from three separate experiments, each conducted in triplicate. *P<0.05 versus control cells (“Cont”) treated with vehicle alone; §P<0.05, significant reversal of the inhibitory effects.

FIG. 4 shows effects of ICI182780 (50 μmol/L) on the inhibitory effects of estradiol ((“β-Est”); 10 nmol/L), 2-hydroxyestradiol ((“2-OHE”); 10 nmol/L), and 2-methoxyestradiol ((“2-MeOE”); 10 nmol/L) on 2.5% FCS-induced ET-1 release into the medium of cells incubated for 4 hours in accordance with methods of the present invention. Values represent mean ±SEM from three separate experiments, each conducted in triplicate. *P<0.05 versus control cells treated with vehicle alone; §P<0.05, significant reversal of the inhibitory effects.

FIG. 5 shows concentration-dependent effects of genistein on 2.5% fetal calf serum-stimulated release of ET-1 in the medium of PCAECs treated for 4 hours in the presence and absence of ICI182780 (50 μmol/L) in accordance with methods of the present invention. Values represent mean ±SEM from three separate experiments, each conducted in triplicate. *P<0.05 versus control cells treated with vehicle alone.

FIGS. 6A-6B show, respectively, concentration-dependent effects of estradiol (“β-Estradiol”), 2-hydroxyestradiol (“2-OH E”), and 2-methoxyestradiol (“2-MeOE”) on 2.5% fetal calf serum (“FCS”)-induced mitogen-activated protein kinase (“MAPK”) activity in PCAECs (FIG. 6A) and effects of ICI182780 (50 μmol/L) on the inhibitory effects of 10 nmol/L estradiol (“β-Est”), 2-hydroxyestradiol (“2-OHE”), and 2-methoxyestradiol (“2-MeOE”) on 2.5% FCS-induced MAPK activity (FIG. 6B) in accordance with methods of the present invention. Results are mean ±SEM (expressed as pmol/min/mg protein). §P<0.05 versus control cells treated with vehicle (“Veh”) alone; §P<0.05, significant reversal of the inhibitory effects.

### DETAILED DESCRIPTION OF THE INVENTION

### I. Definitions

The term “estradiol” refers to 17β-estradiol.

The term “estradiol metabolite(s)” refers to metabolites of 17β-estradiol such as catecholestradiols and methoxyestradiols which exert little estrogenic activity and have a low affinity for the estrogen receptor, examples of which include but are not limited to 2-methoxyestradiol, 4-methoxyestradiol, 2-hydroxyestradiol and 4-hydroxyestradiol.

The term “biodegradable” refers to polymers that dissolve or degrade in vivo within a period of time that is acceptable in a particular therapeutic situation. This time is typically less than five years and usually less than one year after exposure to a physiological pH and temperature, such as a pH ranging from 6 to 9 and a temperature ranging from 25° C. to 40° C.

The term “individual” refers to either a human or animal of the male or female gender.

The term “prodrug” refers to a compound that releases an estradiol metabolite.

### II. Methods

The present invention provides methods of using estradiol metabolites to treat a wide range of conditions including, but not limited to hypertension, heart failure, ischemic heart disease, cerebral vasospasm, renal disease, and migraine headaches or any other health condition where endothelin production is causative or contributory. The invention also provides methods of administering a therapeutically effective amount of a composition containing an estradiol metabolite for the treatment of such disorders.

Estradiol metabolites used to carry out the methods of the present invention include metabolites of 17β-estradiol such as catecholestradiols and methoxyestradiols which exert little estrogenic activity and have low affinity for the estrogen receptor, examples of which include 2-methoxyestradiol, 4-methoxyestradiol, 2-hydroxyestradiol and 4-hydroxyestradiol as well as others. Such estradiol metabolites may be incorporated in a controlled release formulation. Such estradiol metabolites may also be released from prodrugs.

Biodegradable microparticles or nanoparticles that may be used in a controlled release formulation include one or more polymers such as poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes, and blends and copolymers thereof.

### Estradiol Metabolites

Estradiol metabolites used in accordance with the present invention include catecholestradiols such as 2-hydroxyestradiol (estra-1,3,5 (10)-triene-2,3,17-triol (17β)) or 4-hydroxyestradiol (estra-1,3,5 (10)-triene-3,4,17-triol (17β)) or methoxyestradiols, such as 2-methoxyestradiol (estra-1,3,5 (10)-triene-2-methoxy-3,17-diol (17β)) or 4-methoxyestradiol (estra-1,3,5 (10)-triene-4-methoxy-3,17-diol (17β)). Commercial preparations of all of these compounds are readily available.

Estradiol metabolites may also be incorporated into a controlled release formulation. Such controlled release formulations may be biodegradable microparticles, biodegradable nanoparticles, patches, crystals, gels, hydrogels, liposomes, and the like. In addition, the estradiol metabolites may be incorporated into devices, such as implants, vaginal rings, osmotic pumps, diffusion devices and transdermal delivery devices. According to the present invention prodrugs of estradiol metabolites may also be used. Specific examples include esters of hydroxyestradiols and methoxyestradiols.

It will be apparent to the skilled artisan that the compounds listed above are exemplary only and that many variations may be used, depending on the particular hydroxylation or methylation site on the parent estradiol compound. For example, estradiol can be hydroxylated or methylated at many sites and such variations are known in the art.

### Modes of Administration

Therapeutic compositions of the present invention can be formulated in an excipient that the individual to be treated can tolerate. Examples of such excipients include water, saline, Ringer's solution, dextrose solution, Hank's solution, and other physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability.

Examples of buffers include phosphate buffer, bicarbonate buffer and Tris buffer, while examples of preservatives include thimerosal, cresols, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline is added prior to administration.

The present invention also has the objective of providing suitable topical, oral, systemic and parenteral formulations of the pharmaceutical compounds herein provided. The formulations can be administered in a wide variety of therapeutic dosage forms in conventional vehicles for administration. For example, the compounds can be formulated for oral administration in the form of tablets, capsules (each including timed release and sustained release formulations), pills, powders, granules, elixirs, tinctures, solutions, suspensions, syrups and emulsions, or by injection. Likewise, they may also be administered intravenously (both bolus and infusion), during angioplasty/catheterization, intraperitoneally, subcutaneously, topically with or without occlusion, or intramuscularly, all using forms well known to those of ordinary skill in the pharmaceutical arts.

Therapeutic compositions of the present invention can also include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells and glycols.

### Controlled Release Formulations

The method of the present invention can also employ controlled release formulations that are capable of slowly releasing a composition of the present invention into an individual. As used herein, a controlled release formulation can include a composition of the present invention in a controlled release vehicle. Such controlled release formulations are well known in the art. Suitable controlled release formulations include, but are not limited to, biocompatible polymers, other polymeric matrices, capsules, microcapsules, microparticles, nanoparticules, patches (see, U.S. Pat. Nos. 6,238,284; and 5,736,154, the disclosures of which are incorporated herein by reference), crystals (see, U.S. Pat. No. 5,827,531, the disclosure of which is incorporated herein by reference), bolus preparations, liposomes (see, U.S. Pat. Nos. 6,339,069; and 6,143,716, the disclosures of which are incorporated herein by reference), lipospheres, gels (see, U.S. Pat. No. 5,830,506, the disclosure of which is incorporated herein by reference), and hydrogels (see, U.S. Pat. Nos. 6,372,813; 6,372,248; and 6,367,929, the disclosures of which are incorporated herein by reference). Such controlled release vehicles also include devices, such as vaginal rings (see, U.S. Pat. Nos. 6,103,256; and 5,788,980, the disclosures of which are incorporated herein by reference), implants (see, U.S. Pat. Nos. 6,251,418; and 5,874,098, the disclosures of which are incorporated herein by reference), osmotic pumps, diffusion devices, and transdermal delivery systems. Other controlled release formulations of the present invention include liquids that, upon administration to an individual, form a solid or a gel in situ. Preferred controlled release formulations are biodegradable. All such compositions are well known in the art.

A preferred controlled release formulation of the present invention is capable of releasing a composition of the present invention into an individual at a constant rate sufficient to attain therapeutic dose levels of the composition. The therapeutic composition is preferably released over a period of time ranging from 1 day to about 12 months. In a preferred embodiment, such therapeutic composition is released over a 2, 3, 4, 5, 6, 7 day through a 30 day time period.

### Effective Dosage

Acceptable protocols to administer therapeutic compositions of the present invention in an effective manner include individual dose size, number of doses, frequency of dose administration, and mode of administration. Determination of such protocols can be accomplished by those skilled in the art. A suitable single dose is a dose that is capable of protecting (i.e. preventing or treating) an animal or human from disease when administered one or more times over a suitable time period. The need for additional administrations of a therapeutic composition can be determined by one of ordinary skill in the art in accordance with the given condition of a patient.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

In addition, for any compound used in the method of the present invention, the therapeutically effective dose can be estimated initially from cell culture assays. For example, a dose can be formulated in animal models to achieve a circulating concentration range that includes the IC50 as determined in cell culture (i.e., the concentration of the test compound which achieves a half-maximal inhibition of the endothelin activity). Such information can be used to more accurately determine useful doses in humans.

### Treatment of Health Conditions

The present invention provides methods of administering to an individual a therapeutically effective amount of an estradiol to decrease endothelin production. Such estradiol metabolites may be incorporated in a controlled release formulation. They may further be administered in a prodrug formulation. The endothelin production decreased according to the present invention may be endothelin-1.

Health conditions where such decrease in endothelin production may have a therapeutic effect include hypertension, heart failure, ischemic heart disease, cerebral vasospasm, renal disease, and migraine headaches.

In addition, the present inventions as exemplified herein clearly show that many of the beneficial effects of estrogen therapy are mediated by some of the non-feminizing estrogen metabolites and that many of the beneficial effects derived from estrogen therapy depend largely on an individual's ability to metabolize estrogen to the physiologically active and beneficial metabolites. Thus estradiol metabolites may be useful for the treatment and prevention of a broad range of conditions in females who do not adequately and/or correctly metabolize estradiol to the preferred metabolites as well as being useful in post-menopausal women who no longer have endogenous levels of estrogen. Similarly, these non-feminizing estradiol metabolites may be useful for the treatment and prevention of a broad range of conditions in men for whom estradiol therapy, which might otherwise be beneficial, is contraindicated because of the feminizing side-effects.

The present invention will now be further illustrated, but is by no means limited to, the following examples. It will be apparent to those skilled in the art that the techniques described in the examples represent techniques discovered by the inventors to function well in the practice of the invention, and thus can be considered to constitute presently preferred modes for its practice. However, it should be apparent to those of skill in the art that many modifications, both to materials and methods may be made in the specific embodiments without departing from the spirit and scope of this invention.

### EXAMPLES

### Example 1

### β Isolation and Culture of Porcine Coronary Artery Endothelial Cells

Hearts from female pigs were obtained from a local slaughter house and placed in ice-cold oxygenated DMEM (Gibco, Rockville, Md. now Invitrogen, Carlsbad Calif., hereinafter “Gibco”) containing antibiotics (Gibco). The epicardial coronary arteries were isolated, and the fat and connective tissue were removed. Endothelial cells were isolated by collagenase/dispase digestion (Ohbayashi, A., et al., Biochem. Brophys. Res. Commun. 202:504 (1994), the disclosure of which is incorporated herein be reference (“Ohbayashi, et al.”)). The isolated cells were washed in DMEM/F12 medium (Gibco) supplemented with 10% fetal calf serum (“FCS”) (HyClone Laboratories, Logan, Utah) and endothelial cell growth supplement (Clonetics, Walkersville, Md.), and cells were grown to confluence under standard tissue culture conditions. The purity of the cultures, which was characterized by immunostaining with factor VIII antigen (Sigma, St. Louis, Mo.) and by assaying the preferential uptake of the fluorescent probe Dil-acetylated low density lipoprotein (Biomedical Technologies, Inc., Stoughton, Mass.), (Ohbayashi, et al.) was 98%. Cells were passaged by trypsinization, and cells in second and third passage were used for all experiments in this example.

### Treatment Protocols for Endothelin-1 Synthesis Studies

To study the effects of estradiol and its metabolites on ET-1 synthesis in porcine coronary artery endothelial cells (“PCAECs”) (Gibco), confluent monolayers of PCAECs were washed twice with HBSS (Gibco) and treated with DMEM/F12 (gibco) supplemented with 0.4% bovine serum albumin (BSA) containing or lacking 0.001 to 1 μmol/L of estradiol, 2-hydroxyestradiol, or 2-methoxyestradiol (Steraloids, Newport, R.I.). After 24 hours of treatment, the monolayers were treated for an additional 4 hours with fresh treatments in the presence of vehicle, angiotensin II (100 nmol/L)(Sigma), TNF α(10 ng/mL) (Sigma), thrombin (4 U/mL)(Sigma), or FCS (2.5%). The supernatants were collected for ET-1 analysis, and the samples were frozen at −70° C. until analyzed. ET-1 levels were measured with the BIOTRAK™ (Amersham Pharmacia Biotech Europe Gmbh, Dubendorf, Switzerland) high-sensitivity endothelin-1 human ELISA system.

Cells were also treated as above in the presence and absence of the estradiol receptor (“ER”) antagonist, ICI182780 (50 μmol/L; Tocris Cookson Ltd, Ballwin, Mo.). To analyze whether the inhibitory effects of estradiol were mediated by means of ER-β, the effects of genistein (0.001 to 10 μmol/L) (Sigma) were investigated. To evaluate whether the effects of estradiol were mimicked by various clinically used estrogens, the cells were treated as above, in the presence and absence of ICI182780, with 10 nmol/L of estradiol valerate, estradiol cypionate, estradiol benzoate, 17α-estradiol, estrone, estriol or estrone sulfate (all from Sigma), and the ET-1 synthesis in response to FCS (2.5%) was analyzed.

To analyze the effects of estradiol and its metabolites on the basal synthesis of ET-1, cells were pretreated with or without the various above-listed agents and with and without ICI182780 for 24 hours and were fed fresh DMEM/F12 supplemented with 0.4%; BSA (Sigma) and the respective treatments. The supernatants were collected after 4, 8, 12, and 24 hours and the ET-1 levels were analyzed.

### Protocols for MAPK Activity Measurement

PCAECs were grown to confluence in 35-mm² culture dishes and were made quiescent by feeding DMEM containing 0.4% BSA for 48 hours. Growth arrested PCAECs washed with PBS and pretreated for 24 hours with or without various test agents were stimulated with FCS (2.5%) for 10 minutes. Some cells were pretreated for 1 hour with ICI182780 before treatment with the test agents. After stimulation, the cells were washed with ice-cold PBS and extraction buffer (50 mmol/L β-glycerophosphate, 1.5 mmol/L EGTA, 1 mmol/L dithiothreitol, 100 μmol/L Na₃VO₄, 10 μg/mL aprotinin, 5 μg/mL pepstatin, 20 g/mL leupeptin, and 1 mmol/L benzamidine) (all from Sigma), scraped off the plates, and sonicated for 20 seconds in 0.5 mL of extraction buffer. The extracts were collected, the cytosolic fractions were separated by centrifuging the extracts at 100,000×g for 20 minutes at 4° C., and the supernatants were diluted to a concentration of 1 mg protein/mL and stored at −70° C. for mitogen-activated protein kinase (“MAPK”) activity assays. The MAPK activity in the cytosolic extracts was quantified as described by Dubey, R. K., et al., Arterioscler. Thromb. Vasc. Biol. 20:964 (2000), the disclosure of which is incorporated herein by reference.

### III. Results

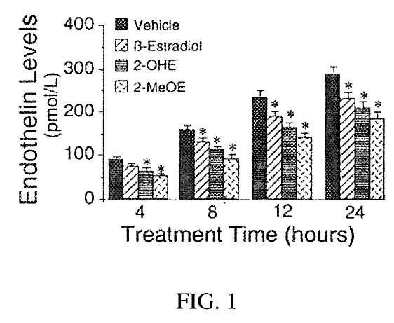

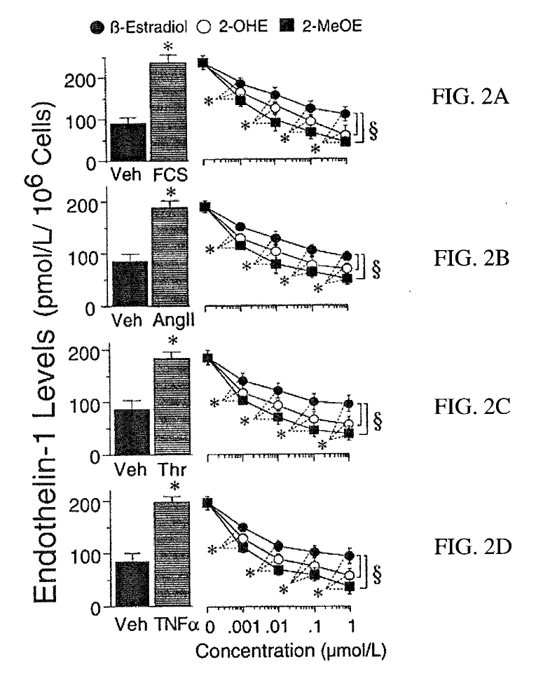

ET-1 levels were increased in a time-dependent manner in the medium collected from PCAECs that were incubated for 4 to 24 hours under basal conditions (FIG. 1). Preliminary studies showed that treatment of PCAECs with FCS stimulated ET-1 secretion maximally at 4 hours, and this time point was selected for all subsequent studies. As compared with vehicle-treated controls, treatment of PCAECs for 4 hours with FCS, angiotensin II, thrombin, and TNFα induced ET-1 secretion by 138±14%, 116±7%, 98±5%, and 120±8% (percentage increase), respectively (FIGS. 2A-2D). Treatment of PCAECs for 8 hours with physiological concentrations (2 nmol/L) of estradiol inhibited basal synthesis of ET-1 by 19±2% (P<0.05). Significant reductions in ET-1 levels were also evident in PCAECs treated under basal conditions for 12 and 24 hours (FIG. 1).

Estradiol inhibited FCS-, angiotensin II-, thrombin-, and TNFα-induced ET-1 secretion in a concentration-dependent manner (FIGS. 2A-2D). Physiological concentrations (1 nmol/L) of estradiol significantly (P<0.05) decreased ET-1 secretion induced in response to FCS (from 138±16% to 82±8%) (FIG. 2A), angiotensin II (from 116±7% to 73±6%) (FIG. 2B), thrombin (from 98±6% to 53±4%) (FIG. 2C), and TNFα (from 120±8% to 68±5%) (FIG. 2D).

The concentration-dependent inhibitory effects of estradiol on basal and stimulated ET-1 secretion were mimicked by its metabolites 2-hydroxyestradiol and 2-methoxyestradiol (FIG. 1 and FIGS. 2A-2D). Compared with estradiol, its metabolites were more effective in inhibiting basal and stimulated ET-1 secretion, the order of potency being 2-methoxyestradiol>2hydroxyestradiol>estradiol (FIGS. 1, and 2A-2D). At physiological concentrations (2 nmol/L), estradiol, 2-hydroxyestradiol, and 2-methoxyestradiol inhibited basal ET secretion in PCAECs incubated for 4, 8, 12, and 24 hours (FIG. 1). Moreover, FCS-, angiotensin II-, thrombin-, and TNFα-induced ET-1 secretion were inhibited by 31%, 33%, 40% and 35% by 2-hydroxyestradiol (1 nmol/L;

FIGS. 2A-2D); by 44%, 39%, 46%, and 56% by 2-methoxyestradiol (1 nmol/L; FIGS. 2A-2D); and by 22%, 20%, 24%, and 26% by estradiol (1 nmol/L; FIGS. 2A-2D).

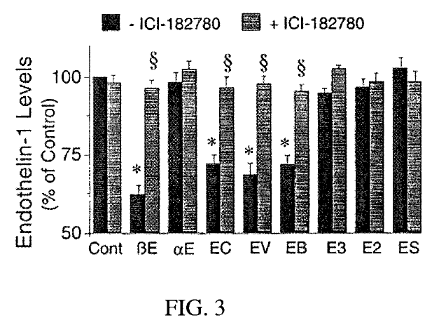

Similar to estradiol, FCS-induced ET-1 synthesis was inhibited by estradiol valerate, estradiol cypionate, and estradiol benzoate (FIG. 3). In contrast, estrone, estrone sulfate, estriol, and 17α-estradiol were unable to lower ET-1 secretion. For all the tested estrogens, the potency order for inhibition of ET-1 synthesis was as follows: estradiol>estradiol valerate=estradiol cypionate>estradiol benzoate>estrone=estriol=estrone sulfate=17α-estradiol.

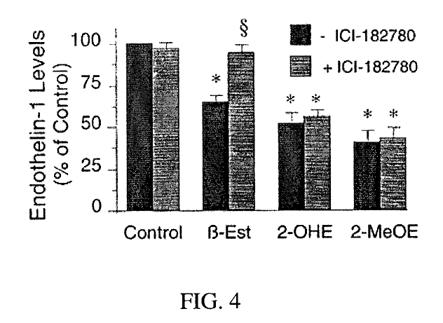

Treatment of PCAECs with 50 μmol/L ICI182780 did not influence basal or FCS-stimulated ET-1 synthesis. ICI182789 completely blocked the inhibitory effects of estradiol, but not 2-hydroxyestradiol and 2-methoxyestradiol, on FCS-stimulated ET-1 secretion (FIG. 4), as well as on basal ET-1 release (data not shown). Similar to estradiol the inhibitory effects of estradiol valerate, estradiol cypionate, and estradiol benzoate were reversed by ICI182780 (FIG. 3).

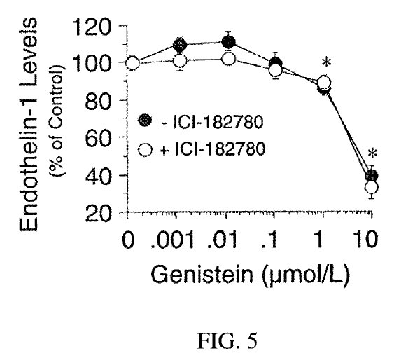

Genistein, an ER-β ligand at nanomolar concentrations, (see, Kuiper, G. G. J. M., et al., Endocrinology 139:4252 (1998), the disclosure of which is incorporated herein by reference) inhibited basal (data not shown) as well as FCS-stimulated (FIG. 5) ET-1 secretion by PCAECs in a concentration-dependent manner. The inhibitory effects of genistein were observed at concentrations of 1 μmol/L and higher, moreover the inhibitory effects of genistein were not reversed by ICI182780 (FIG. 5).

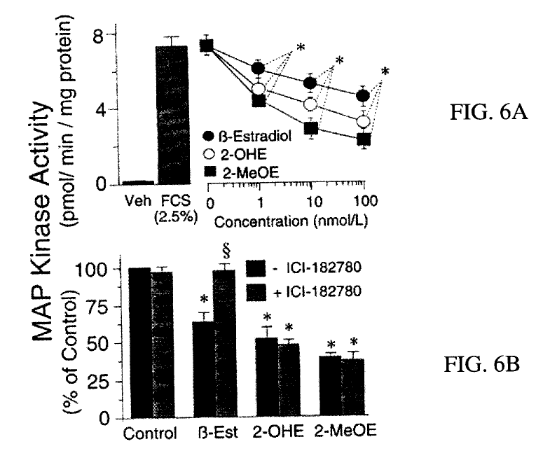

Treatment of growth-arrested cells with FCS (2.5%) increased MAPK activity from 0.187 to 7.25 μmol/min/mg protein, and the stimulatory effects of FCS were inhibited by the MEK inhibitor PD98059 (10 μmol/L) to 0.7 μmol/min/mg protein. In cells pretreated for 24 hours with estradiol, 2hydroxyestradiol, or 2-methoxyestradiol, the stimulatory effects of FCS on MAPK activity were inhibited in a concentration-dependent manner (FIG. 6A). Compared with estradiol, 2-hydroxyestradiol and 2-methoxyestradiol were more potent in inhibiting FCS-induced MAPK activity. In cells pretreated with ICI182780 (50 μmol/L), the inhibitory effects of estradiol, but not 2-hydroxyestradiol or 2-methoxyestradiol, on MAPK activity were completely reversed (FIG. 6B). In PCAECs pretreated for 24 hours with physiological concentrations (1 nmol/L) of estradiol, 2-hydroxyestradiol, and 2-ethoxyestradiol, FCS-induced MAPK activity was inhibited by 19±2.6%, 34±4%, and 46±4.7%, respectively.

While the disclosed methods and compositions have been described in terms of the specific embodiments of the invention, it will be apparent to those of ordinary skill in the art that variations may be applied without departing from the concept, spirit and scope of the claimed invention. More specifically, it will be apparent that certain agents that are both chemically and physiologically related may be substituted for the agents described herein while the same or similar results would be achieved. All such similar substitutes and modifications apparent to those skilled in the art are deemed to be within the invention as defined by the appended claims. Accordingly, it is understood that the drawings and the descriptions herein are proffered only to facilitate comprehension of the invention and should not be construed to limit the scope hereof.

1-5. (canceled)

6-10. (canceled)

11. A method for reducing endothelin production in an individual in need thereof, comprising:
administering to said individual a composition selected from the group consisting of 2-hydroxyestradiol, 2-methoxyestradiol, 4-hydroxyestradiol and 4-methoxyestradiol in order to cause a reduction in endothelin production in said individual.

12. The method of claim 11, wherein said reduction in endothelin production has a therapeutic effect in a health condition selected from the group consisting of hypertension, heart failure, ischemic heart disease, cerebral vasopasm, renal disease and migraine headaches.

13. The method of claim 11, further comprising a prodrug of said estradiol metabolite.

14. The method of claim 11, wherein said composition comprises a controlled release formulation.

15. The method of claim 11, wherein said endothelin production is endothelin-1 production.
